(19) United States
(12) Patent Application Publication
(10) Pub. No.: US 2025/0257025 A1
(43) Pub. Date: Aug. 14, 2025

(54) PROCESS FOR THE PRODUCTION OF BIOACETIC ACID AND USES THEREOF

(71) Applicant: BIOSIMO AG, ZÜRICH ETH-HÖNGGERBERG (CH)

(72) Inventors: EMILY LUPPARELLI, ZÜRICH (CH); MAXIMILIAN MOSER, ZÜRICH (CH)

(21) Appl. No.: 19/051,424

(22) Filed: Feb. 12, 2025

(30) Foreign Application Priority Data
Feb. 12, 2024 (EP) 24157004.3

(51) Int. Cl. C07C 51/235; C07C 51/46

(52) U.S. Cl. CPC C07C 51/235; C07C 51/46

(57) ABSTRACT

The invention relates to a method for the preparation of bio-acetic acid and the product thereof.

### FIELD OF THE INVENTION

The present invention pertains generally to the field of bio-acetic acid production, in particular useful in the food, pharmaceutical, chemical industries.

### BACKGROUND OF THE INVENTION

Acetic acid is commonly used as a reagent to manufacture chemicals such as vinyl acetate monomer, acetate esters, purified terephthalic acid, acetic anhydride.

Traditionally, acetic acid is industrially produced via an indirect route, carbonylation of methanol, using syngas (primarily formed by CO and H₂). Globally, three main environmental drawbacks are found in this synthesis pathway: a) it is an energy-intensive process, b) syngas is mainly produced from fossil fuels (e.g., carbon and natural gas) and c) environmental impacts coming from many chemical waste discharges (Juan Luis Martín-Espejo, 840, 156663).

Therefore, efforts are now concentrated on promoting alternatives to this process. Furthermore, the current tendency in manufacture is claiming a shift to more sustainable chemical routes to lessen the environmental impacts to the atmosphere and hydrosphere of these large-scale industrial processes. Therefore, the alternative bio-routes proposed, using biogas as source, could be considered attractive. However, the bio-production of acetic acid relies on fermentation processes, which produce acetic acid in very low concentrations (<20 wt. %). No catalytic process can convert liquid-diluted bioethanol to bio-acetic acid with a high process yield.

Ethyl acetate is a sustainable alternative for extracting 10-30% wt. % acetic acid in water concentrations (Kürüm et al-. 1995 Chemical Engineering Communications, 136:1, 161-176, DOI: 10.1080/00986449508936359). However, it does not yield the best extraction efficiency and solvent recovery results. Ethyl acetate when used as an extractant with acetic acid can only work with up to 30 wt. % aqueous acetic acid, limiting its use. Furthermore, the use of ethyl acetate to extract acetic acid leads to an exchange of acetate molecules, which can alter the C¹⁴ pMC of the bio-based acetic acid should the ethyl acetate extract not be 100% bio-based itself.

The use of isopropyl acetate has been also described for purifying acetic acid from feedstock solutions of acetic acid having an acetic acid concentration of from 10 to 50% by weight into an extractor (EP5662780) but it has been observed that those processes are limited in yields when inlet concentrations of more than 25% wt. % acetic acid concentrations are used.

Therefore, fermentation cannot scale to compete with the fossil route because of low yields and high dilution. Niche markets are also taken by the two-step ethanol-to-acetic acid gas reaction, which suffers from the same drawbacks. Downstream purification for diluted streams is performed via conventional extraction and distillation processes. Acetic acid extraction is industrially performed via MTBE (Methyl tert-butyl ether) and ethyl acetate, respectively a toxic and non-toxic chemical. The extract is then sent to distillation, where acetic acid is recovered as a high-boiling product. Due to its persistence, MTBE is a long-term hazard to the environment in contrast to ethyl acetate that decomposes to ethanol and acetic rendering it bio-degradable.

Due to the raising demand for a use of bio-acetic acid in the food, pharmaceutical, and chemical sectors, there is a need for the development of new efficient methods for the production of bio-acetic acid in high yields.

### SUMMARY OF THE INVENTION

A general object of this invention is to provide a method of production of bio-acetic acid.

One of the specific objects of this invention is to provide a method of production of bio-acetic acid with high yield.

It is advantageous to provide a method of production of bio-acetic acid which is fully bio-based.

It is advantageous to provide a method of production of bio-acetic acid allowing working with higher acetic acid inlet concentrations.

It is advantageous to provide a method of production of bio-acetic acid allowing to recover valuable byproducts specific to the bioprocess (e.g. acetaldehyde, ethyl acetate, acetal) and recover heavy metal impurities from the acetic acid.

It is advantageous to provide a method of production of bio-acetic acid using less extraction solvent quantities.

It is advantageous to provide a method of production of bio-acetic acid avoiding the formation of C1 molecules like CO₂, CO, and formaldehyde and formic acid.

It is advantageous to provide a method of production of bio-acetic acid using less energy.

It is advantageous to provide a method of production of bio-acetic acid using a solvent which can be produced sustainably from biomass or biowaste.

In particular, it is advantageous to provide a method of production of bio-acetic acid using a solvent which is degradable and does not impact the pMC value of the bio-based acetic acid during extraction.

It is advantageous to provide a method of production of bio-acetic acid using a solvent which has a high miscibility gap with water to allow extracting aqueous acetic acid from concentrations above 30 wt. % acetic acid.

An object of this invention is to provide bio-acetic acid which is produced by a fully bio-based process.

It is advantageous to provide bio-acetic acid with after synthesis levels in C1 molecules such as formaldehyde and formic acid below 10 ppm before any purification and with a C14 pMC equal to the contemporary ASTM reference for bio-based materials.

Objects of this invention have been achieved by providing a method for the preparation of bio-acetic acid according to claim 1 and a method for the preparation of feedstock aqueous solution of acetic acid for use in a method according to claim 1. Objects of the invention have been also achieved by providing a bio-acetic acid according to claim 15.

Disclosed herein is a method for the preparation of bio-based acetic acid comprising the steps of:
a) Providing a feedstock aqueous solution of acetic acid having an acetic acid concentration from about 1 to about 50 wt. % into an extraction vessel;
b) Supplying the feedstock aqueous solution with an extracting medium containing butyraldehyde to separate from the obtained mixture between the extracting medium and the aqueous solution of acetic acid, a water-poor phase rich in butyraldehyde and a water-rich phase;
c) Subjecting the extracted water-poor phase rich in butyraldehyde and acetic acid to an azeotropic distillation to separate acetic acid from butyraldehyde;
d) Recovering anhydrous acetic acid by distillation of the water-poor phase rich in butyraldehyde;
e) Recovering purified water by distillation of the water-rich phase.

Also disclosed herein is a process for the preparation of a bio-based feedstock aqueous solution of acetic acid having an acetic acid concentration from about 1 to about 90 wt. % (e.g. 5 to 90 wt. % or 1 to 50 wt %) comprising the steps of:
Feeding an ethanol/water mixture containing from about 10 to 95 wt. % ethanol into a reaction vessel;
Subjecting the ethanol/water mixture to aerobic oxidation under a temperature from about 160 to about 250° C. (e.g. 160 to 220° C. for example 200° C.) at a pressure of 20-60 bar in presence of a heterogeneous catalyst;
Recovering the reaction products containing acetic acid, water, unreacted ethanol, traces of acetaldehyde, unreacted oxygen, nitrogen and minor impurities (e.g. acetyls, acetates);
Removing the volatiles by evaporation and subjecting the remaining liquid phase to a distillation step for the removal of ethanol and traces of acetaldehyde;
Recovering the aqueous solution from the distillation step, wherein said aqueous solution comprises acetic acid having a concentration from about 1 to about 90 wt. % (e.g. 5 to 90 wt. % or 1 to 50 wt. %), typically 10 to about 50 wt. %, in particular 25-45 wt % (e.g. 30-40 wt. %);
Subjecting the distillates obtained from the distillation step to an evaporation step to recover the unreacted ethanol in water).

Also disclosed herein is a fully bio-based method for the preparation of bio acetic acid. Also disclosed herein is a bio-acetic acid obtainable from a method according to the invention.

Also disclosed herein is a bio-acetic acid with after synthesis levels in formaldehyde and formic acid below 10 ppm before any purification and with a C14 pMC equal to the contemporary ASTM reference for bio-based materials.

Other features and advantages of the invention will be apparent from the claims, detailed description, and figures.

### BRIEF DESCRIPTION OF THE DRAWINGS

FIGS. 1A and 1B are illustrative workflows of the main steps of methods according to the invention. FIG. 1A: method for the preparation of bio-based acetic acid from a feedstock aqueous solution of acetic acid having an acetic acid concentration from about 10 to about 50% wt. %; FIG. 1B: a method for the preparation of feedstock aqueous solution of acetic acid having an acetic acid concentration from about 10 to about 50% wt. % for use in the method for the preparation of bio-based acetic.

FIG. 2 provides extraction ternary diagrams of butyraldehyde (top) compared to ethyl acetate (bottom). The ternary diagrams are modelled with process simulation software CHEMCAD™ that uses thermodynamic properties from its data bank.

FIGS. 3A-3C provide examples of a process flow set-up of a method according to the invention as described in Example 3. FIG. 3A: Detailed exemplary flowsheet of a method for the preparation of feedstock aqueous solution of acetic acid having an acetic acid concentration from about 10 to about 50% wt. % as described in Example 1 for use in the method for the preparation of bio-based acetic acid according to the invention (dotted zone further detailed in B); FIG. 3B: Detailed exemplary flowsheet for the preparation of bio-based acetic acid from a feedstock aqueous solution of acetic acid having an acetic acid concentration from about 10 to about 50% wt. % as described in Example 1 obtained by a method exemplified in FIGS. 1B and 3A (dotted zone from FIG. 3A detailed in FIG. 3B). The numbers indicate the corresponding stream as described in Examples 1 and 3; FIG. 3C: legend of the symbols elements in FIG. 3A and FIG. 3B.

### DETAILED DESCRIPTION OF EMBODIMENTS OF THE INVENTION

“Bio-based” refers to any chemical that has a C¹⁴ isotope level that according to the ASTM D6866 method matches the contemporary C¹⁴ reference value, i.e. for 2024 the C14 pMC is 99.7.

As used herein the expression of “rich” for a phase obtained by phase extraction means containing 90% or more of the referred component. For example, butyl-aldehyde-rich or water-rich phase contains 90% or more of butyl-aldehyde or water, respectively. The “poor” phase as a consequence is the other phase.

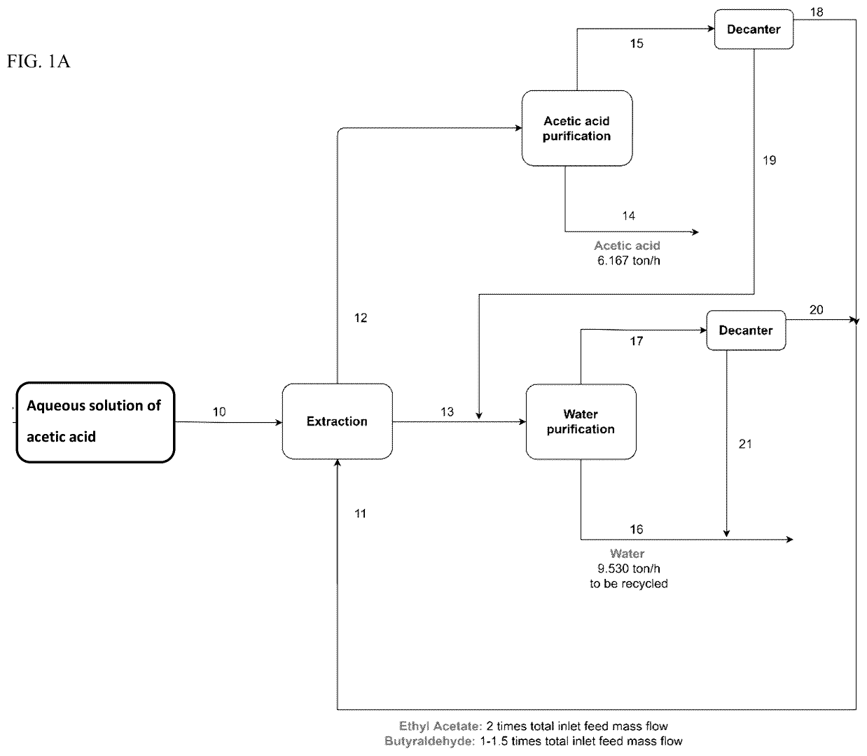

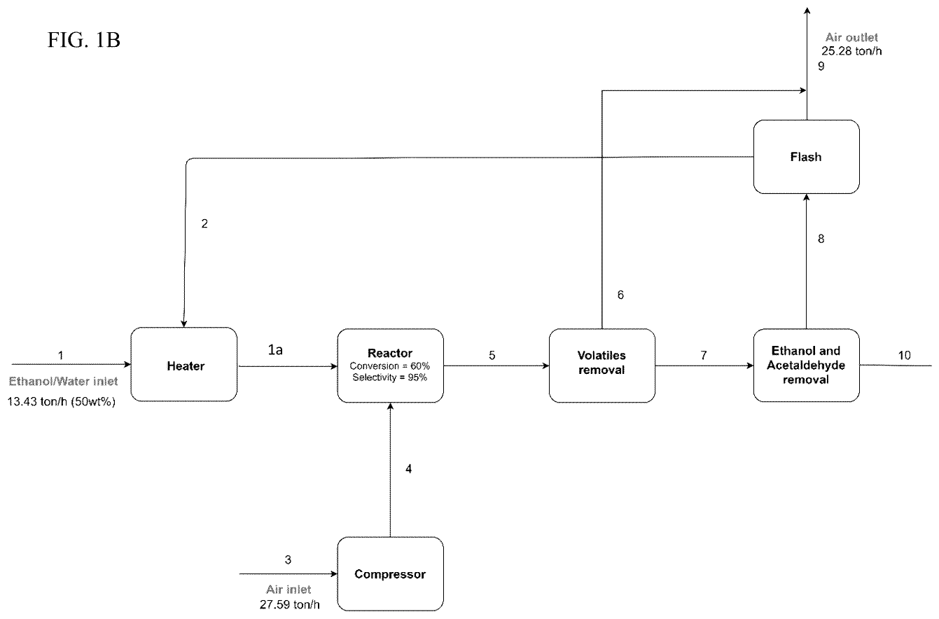

Referring to the figures, in particular first to FIG. 1A, is provided an illustration of a method for the preparation of bio-based acetic acid.

More specifically, the steps of the embodiment illustrated in FIG. 1A comprise:
a) Providing a feedstock aqueous solution of acetic acid having an acetic acid concentration from about 1 to about 50 wt. % into an extraction vessel (stream 10);
b) Supplying the feedstock aqueous solution with an extracting medium containing butyraldehyde to separate from the obtained mixture between the extracting medium and the aqueous solution of acetic acid, a water-poor phase rich in (i.e. 10 wt % of water or less and 90 wt % of butyraldehyde or more) (stream 12) and a water-rich phase (i.e. 90 wt % of water or more) (stream 13);
c) Subjecting the extracted water-poor phase rich in butyraldehyde (i.e. 90 wt % of butyraldehyde or more) (stream 12) and acetic acid to an azeotropic distillation to separate acetic acid from butyraldehyde;
d) Recovering anhydrous acetic acid by distillation of the water-poor phase rich in butyraldehyde (stream 14);
e) Recovering purified water by distillation of the water-rich phase (i.e. 90 wt % of water or more) (stream 16).

According to a particular embodiment, a feedstock aqueous solution of acetic acid having an acetic acid concentration from about 10 to about 50 wt. %. is provided into the extraction vessel.

According to a particular embodiment, butyraldehyde is provided at a mass ratio with the feedstock mass from 1 to 2.

According to a particular embodiment, butyraldehyde is provided at a mass flow rate which is 1 to 2 times the feed mass flow rate.

According to a further embodiment, the butyraldehyde can be produced via the dehydrogenation of butanol or hydrogenation of crotonaldehyde such as described in Jyothi et al., 2014, Indian Journal of Chemistry 53A, 553-556, and Raff, Donald K. Ullmann's Encyclopedia of Industrial Chemistry-Butanals, 2000, respectively. In particular butanol produced from biomass or biowaste such as described in Grim et al. 2019, ACS 9, 5, 4145-4172, 10.1021/acscatal.8b03945; Li et al, 2020, Chemistry Select, 5, 28, 8669-8673; doi.org/10.1002/slct.202001063 or Gabriels et al., 2015, Catalysis, Science & Technology, 8, 10.1039/C5CY00359H.

According to a particular embodiment, the method can be carried out in a continuous or semi-continuous flow.

According to a more particular embodiment, when the method is carried out in a continuous or semi-continuous flow, the feedstock aqueous solution is provided at a flow rate from about 1 ton/h to about 100 ton/h into an extraction vessel.

According to a particular embodiment, the feedstock aqueous solution is provided at a temperature from about 20 to about 25° C. (e.g. 25° C.) and at atmospheric pressure to the extraction vessel.

According to another particular embodiment, when the method is carried out in a continuous or semi-continuous flow, the extracting medium containing butyraldehyde is supplied at a flow rate from about 1 ton/h to about 200 ton/h into an extraction vessel.

According to another particular embodiment, the extraction vessel is a 10-30-stage extraction column, typically 20.

According to a particular embodiment, the obtained mixture between the extracting medium and the aqueous solution of acetic acid is subjected to an extraction in the extraction vessel at a temperature from about 10-35° C., typically for about 20 to 25° C. to separate water-poor phase rich in butyraldehyde and acetic acid from a water-rich phase.

According to a particular embodiment, the extracted water-poor phase rich in butyraldehyde is subjected to an azeotropic distillation at a temperature from about 50-150° C., typically for about 70-120° C., under atmospheric pressure to separate acetic acid from butyraldehyde.

According to a particular embodiment, the azeotropic distillation is carried out in an azeotropic column which operates at 50-150° C., typically at 70-120° C., such as 71-117° C.

According to another particular embodiment, when the method is carried out in a continuous or semi-continuous flow, the water-poor phase rich in butyraldehyde is supplied to a distillation column at a flow rate from about 5 ton/h to about 120 ton/h.

According to another particular embodiment, the bottom liquid phase obtained as bottom product from the azeotropic distillation column contains anhydrous acetic acid with an amount from about 99.8 and 99% wt. of said phase (e.g. 99.85% wt.).

According to another particular embodiment, the distillate containing butyraldehyde is obtained as distillate product from the azeotropic distillation column, wherein said distillate contains from 1 to 20 wt. % water (e.g. 10 wt. %) and 80 to 99 wt. % (e.g. 90 wt. %) butyraldehyde.

According to another particular embodiment, the butyraldehyde is further extracted from the distillate extracted from distillation column. For example, the distillate containing butyraldehyde (stream 15) is subjected to a decanter to isolate butyraldehyde from remaining water. In this case, the water extracted from the decanter can be then fed to the water-rich phase (Stream 13) for further purification and the butyraldehyde recovered from the decanter (Stream 18) can be then recycled into the feedstock aqueous solution as extracting medium (Stream 11).

According to a particular embodiment, the distillation of the water-rich phase is carried out at a temperature from about 50-120° C., typically for about 70-100° C.

According to another particular embodiment, the water-rich phase obtained from the azeotropic distillation (Stream 13) is supplied to a water purification system.

According to a more particular embodiment, the water purification system is a water purification column which operates at a temperature range of typically 50-120° C., typically 70-100° C., under atmospheric pressure.

According to another particular embodiment, when the method is carried out in a continuous or semi-continuous flow, the water-rich phase obtained from the extraction column is supplied at a flow rate from about 2 ton/h to about 40 ton/h to the water purification column.

According to another particular embodiment, the purified water recovered from the water purification column and can be recycled.

According to another particular embodiment, the distillate recovered from the water purification column (stream 17) containing low-boiling impurities is subjected to a decanter from which purified water can be extracted (stream 21).

According to another particular embodiment, water-rich phase (Stream 21) can be recycled back to the reaction inlet.

According to another particular embodiment, the decanter used for the purification of the distillate from the water purification column operates at a pressure of 1-10 bar and a temperature of 10-30° C., typically at 1-5 bar and 20-25° C.

According to a further embodiment, the solvent-phases recovered from each of the decanters (streams 18 and 20) are recycled back to the extraction vessel (stream 11).

According to a further embodiment, the solvent impurities in water, recovered by distillation of the water-rich phase, can be recycled back to the extraction vessel (stream 19).

According to a further embodiment, feedstock aqueous solution of acetic acid having an acetic acid concentration from about 10 to about 50% wt. % is prepared by catalytic oxidation of liquid bioethanol with air or enriched oxygen as described herein or in WO 2021/239641.

According to a particular embodiment, the feedstock aqueous solution of acetic acid having an acetic acid concentration from about 1 to about 90 wt. % (e.g. 10 to 50 wt %) is prepared by a method comprising the following steps:
Feeding an ethanol/water mixture containing from about 10 to 95 wt. % ethanol into a reaction vessel;
Subjecting the ethanol/water mixture to aerobic oxidation under a temperature from about 160 to about 250° C. at a pressure of 20-60 bar in presence of a heterogeneous catalyst;
Recovering the reaction products containing acetic acid, water, unreacted ethanol, traces of acetaldehyde, unreacted oxygen, nitrogen and minor impurities (e.g. acetyls, acetates);
Removing the volatiles by evaporation and subjecting the remaining liquid phase to a distillation step for the removal of ethanol and traces of acetaldehyde;
Recovering the aqueous solution from the distillation step, wherein said aqueous solution comprises acetic acid having at a concentration from about 1 to about 90 wt. % (e.g. 5 to 90 wt. % or 1 to 50 wt. %), typically 10 to about 50 wt. %, in particular 25-45 wt % (e.g. 30-40 wt. %);
Subjecting the distillates obtained from the distillation step to an evaporation step to recover the unreacted ethanol in water).

According to a particular aspect, bio-acetic acid prepared by the above method contains after synthesis and before any purification (stream 5), levels in C1 molecules such as formaldehyde and formic acid below 10 ppm and with a C14 pMC equal to the contemporary ASTM reference for bio-based materials.

According to a particular aspect, the ethanol/water mixture is a bioethanol mixture obtainable by bioethanol production processes such as fermentation of biomass, biowaste or form captured CO₂.

According to a particular aspect, the aerobic oxidation comprises supplying the reaction vessel with air flow with an amount of oxygen that is 0.5-1 to 2 times the molar content in ethanol of the mixture and raising the pressure of the reaction vessel to reach a pressure of 20-60 bar, typically 30-50 bar.

According to a particular aspect, the aerobic oxidation can be conducted under air, oxygen-enriched air, or oxygen.

According to a particular aspect, the ethanol/water mixture is heated at a temperature from about 160 to about 250° C. before entering into contact with the catalyst to maximize the catalyst efficacy.

According to a particular aspect, the ethanol/water mixture is heated at a temperature from about 160 to about 250° C. in a heating vessel before entering into contact with the catalyst in a reaction vessel.

According to a particular aspect, the ethanol/water mixture is heated at a temperature from about 160 to about 250° C. in a reaction vessel before entering into contact with the catalyst in said reaction vessel.

According to a particular aspect, the method for the preparation of feedstock aqueous solution of acetic acid having an acetic acid concentration from about 10 to about 50% wt. % according to the invention yields to more than 90% Selectivity and >60% conversion (exact value depending on reactor size). Typically, 95% selectivity to acetic acid and >60% conversion of ethanol is obtained in single-pass at 160 to 230° C. at 30-50 bars for 1 g to 1000 g heterogeneous catalyst in single tube.

Referring to the figures, in particular first to FIG. 1B, is provided an illustration of a method for the preparation of feedstock aqueous solution of acetic acid having an acetic acid concentration from about 10 to about 50% wt. % for use in the method for the preparation of bio-based acetic acid as illustrated in FIG. 1A.

More specifically, the steps of the embodiment illustrated in FIG. 1B comprise:
i) Feeding an ethanol/water mixture containing from about 10 to 95 wt. % ethanol into a heating vessel (stream 1);
ii) Bringing the temperature of the ethanol/water mixture to a temperature from about 160 to about 250° C. (typically 175-190° C. e.g. 180° C.);
iii) Supplying the heated ethanol/water mixture to a heterogeneous catalyst at a temperature from about 160 to about 250° C.;
iv) Supplying the heated ethanol/water mixture in presence of the heterogeneous catalyst with air, oxygen-enriched air, or oxygen flow with an amount of oxygen that is 0.5-1.25 times the molar content in ethanol of the mixture and raising the pressure of the reaction vessel to reach a pressure of 20-60 bar, typically 30-50 bar (stream 4) to trigger an aerobic oxidation of the heated ethanol/water mixture;
v) Recovering the reaction products (stream 5) containing acetic acid, water, unreacted ethanol, traces of acetaldehyde, unreacted oxygen, nitrogen and minor impurities (e.g. acetyls, acetates);
vi) Removing the volatiles (e.g. oxygen, nitrogen, and acetaldehyde) by evaporation (stream 6) and transferring the remaining liquid phase (Stream 7, mainly containing acetic acid, water and unreacted ethanol) to a distillation step for the removal of ethanol and traces of acetaldehyde;
vii) Recovering the aqueous solution from the distillation step (stream 10), wherein said aqueous solution comprises acetic acid having at a concentration from about 5 to about 90% wt. %, for example 10 to about 50 wt. %, typically 25-45 wt % (e.g. 30-40 wt. %);
viii) Subjecting the distillates (stream 8) obtained from the distillation step to an evaporation step to recover the unreacted ethanol in water (e.g. with a concentration of 50-99%, typically 70-85%) (stream 2).

According to a particular aspect, the flow rate and composition of Stream 1 are adjusted based on the flow rate and composition of Stream 2 to match the desired inlet composition

According to a particular embodiment, the heterogeneous catalyst is selected from ruthenium oxide-based, gold-based, platin-based, palladium-based, tungsten oxide-based and vanadium oxide-based catalyst.

According to a more particular embodiment, the heterogeneous catalyst is a ruthenium oxide-based catalyst.

According to a particular aspect, the aerobic oxidation is carried out in a trickle bed reactor comprising a heterogeneous catalyst bed (e.g. as described in WO 2021/239641) wherein ethanol in a concentration of 10 to 95% in water is reacted with oxygen, oxygen-enriched air or air over a catalyst bed (e.g. RuO₂-based) to produce acetic acid at pressures of 20 to 60 bars and temperatures of 160 to 250° C. to produce acetic acid.

According to a particular embodiment, the reaction products (stream 5) containing acetic acid, water, unreacted ethanol, traces of acetaldehyde, unreacted oxygen, nitrogen and minor impurities (e.g. acetyls, acetates) are supplied into a first flash evaporation vessel at 1-10 bar, typically 1-5 bar, and 12-25° C., typically 18-20° C. from which the volatiles can be removed by evaporation at a temperature from about 50-120° C., typically 75-102° C.

According to another particular embodiment, the distillates from the distillation step can be transferred to a second flash evaporation at 1-10 bar, typically 1-5 bar, and 12-25° C., typically 18-20° C. to recover the unreacted ethanol in water with a concentration of 50-99% wt. %, typically 70-85% wt. % (stream 2) which can be recycled into the heating vessel.

According to a particular embodiment, unreacted ethanol recovered from the distillation step is recycled into the heating vessel.

According to a particular embodiment, the method for the preparation of feedstock aqueous solution of acetic acid according to the invention can be carried out in a continuous or semi-continuous flow.

According to a particular aspect, the aqueous solution of acetic acid recovered from the above method has an acetic acid concentration from about 10 to about 50% wt. % and can be used as a feedstock for the preparation of bio-based acetic acid according to a method of the invention.

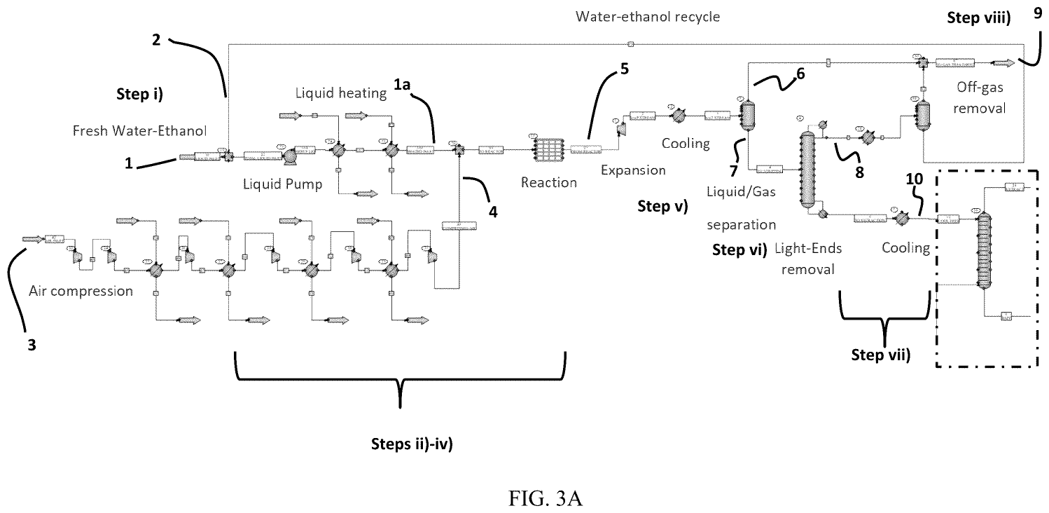

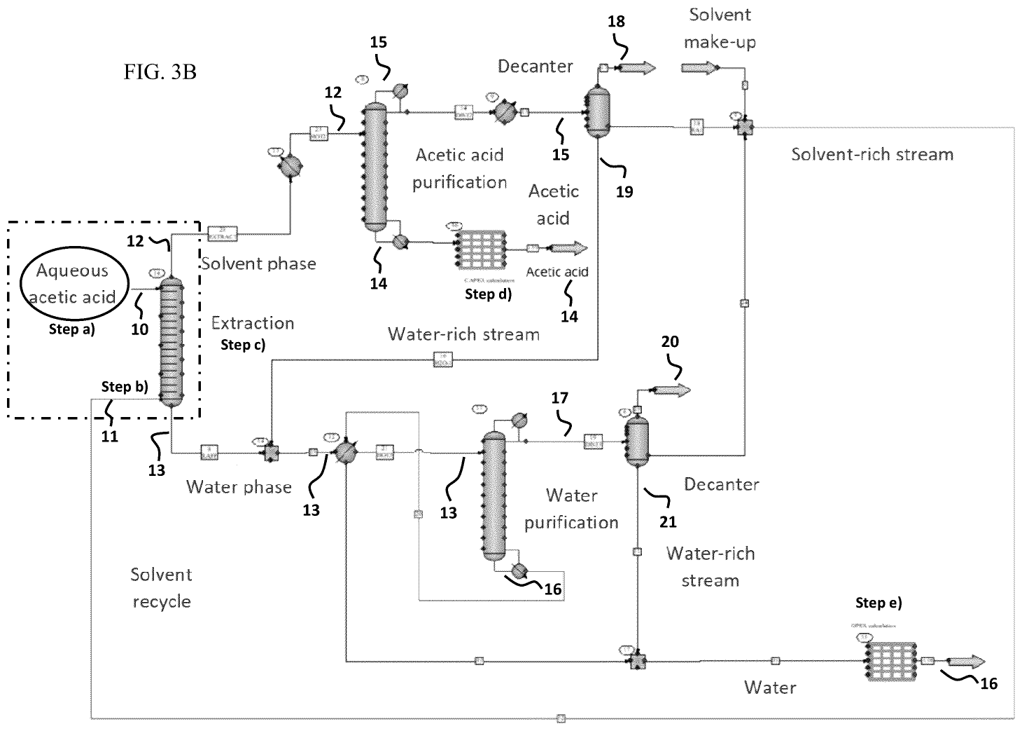

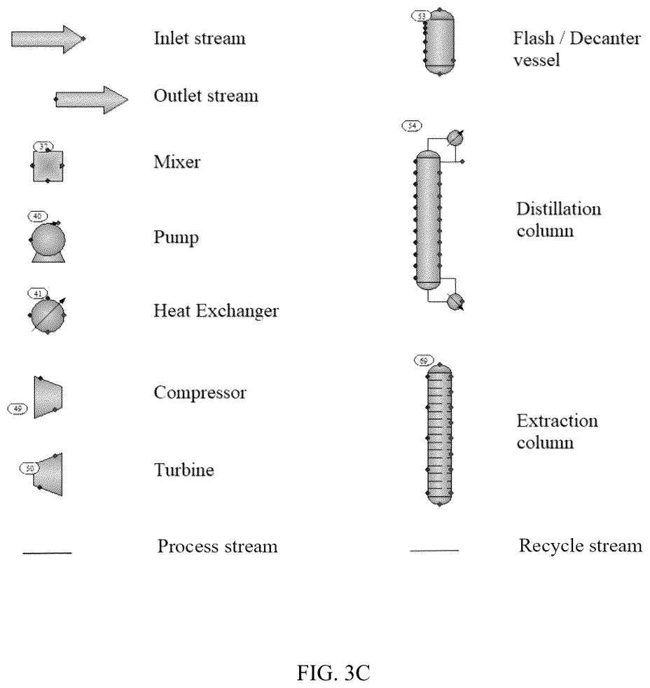

FIGS. 3A & B, provide an illustration of a set-up for the preparation of bio-based acetic acid according to a method of the invention.

The invention having been described, the following examples are presented by way of illustration, and not limitation.

### EXAMPLES

### Example 1: Method for the Preparation of Bio-Based Acetic Acid According to the Invention

A method of the invention was carried out as an Example as illustrated in FIG. 1A.
a) Providing a feedstock aqueous solution of acetic acid having an acetic acid concentration from about 1 to about 50% wt. % into an extraction vessel

An acetic acid/water mixture (between 10% and 50 wt. %) is provided into an extraction column being a Kühni column of 3 m of diameter and 14 m of height. Example for a 50-kiloton plant: 15.68 ton/h of 39.32% acetic acid in water are fed to a 20-stage extraction column. The mixture stream (Stream 10) is at atmospheric pressure (1 atm) and is cooled down to 25° C. before entering the extraction column.
b) Supplying the feedstock aqueous solution with an extracting medium containing butyraldehyde to separate from the obtained mixture between the extracting medium and the aqueous solution of acetic acid, a water-poor phase rich in butyraldehyde (stream 12) and a water-rich phase (stream 13)

An extracting medium containing butyraldehyde in an amount corresponding to the mass flow of the acetic acid/water mixture is added to the acetic acid/water mixture. For this purpose, a butyraldehyde stream of 16.56 ton/h (3.5% Water; 96.5% Butyraldehyde) at 25.85° C. and 1 bar is introduced at the bottom of the extraction column.
c) Subjecting the extracted water-poor phase rich in butyraldehyde (stream 12) and acetic acid to an azeotropic distillation to separate acetic acid from butyraldehyde

The extracted water-poor phase is then subjected to an azeotropic distillation at a temperature of 70-120° C.
d) Recovering anhydrous acetic acid by distillation of the water-poor phase rich in butyraldehyde

The water-poor phase rich in butyraldehyde and acetic acid is supplied to a distillation column at a temperature of 70-120° C. under atmospheric pressure to separate acetic acid from butyraldehyde. The number of selected theoretical stages is 24, feed stage is 10, and the molar reflux ratio is 1.8. The feed of the water-poor phase rich in butyraldehyde and acetic acid (Stream 12) was provided to the distillation column at a feed rate of 23.62 ton/h (7.37% water, 26.1% acetic acid, 66.5% butyraldehyde) under a pressure of 1 bar and at a temperature maintained at 35° C. The bottom phase containing anhydrous acetic acid is extracted in the outlet bottom stream of the column (Stream 14) at a rate of 6.167 ton/h of 99.8% acetic acid at 117.3° C. and 1 bar. The distillate containing butyraldehyde is extracted from the column (Stream 15) is a rate 17.44 ton/h stream (9.91% water and 90.09% butyraldehyde) at 70.77° C. and 1 bar and further subjected to a decanter to isolate butyraldehyde from remaining water.

The water extracted from the decanter (Stream 19) is then fed with the water-rich phase (Stream 13) and butyraldehyde extracted from the decanter (Stream 18) is then fed to the feedstock aqueous solution as extracting medium (Stream 11).
e) Recovering purified water by distillation of the water-rich phase

The water-rich phase (Stream 13) is supplied to a water purification column a temperature range of 70-100° C., under atmospheric pressure. The number of selected theoretical stages is 6, feed stage is 2, and the molar reflux ratio is 0.4. The feed of water-rich phase (Stream 13+Stream 19) is provided to the water purification column at a rate of 9.821 ton/h stream (3.1% butyraldehyde, 96.9% water) at 1 bar under a temperature of 55° C. Purified water is recovered in the outlet bottom stream (Stream 16) of the water purification column at a rate of 9.442 ton/h of 99.99% water at 99.59° C. and 1 bar. The distillate (Stream 17) is collected from the water purification at a rate of 0.379 ton/h (20.1% water and 79.9% butyraldehyde) at 72.96° C. and 1 bar and further subjected to a decanter to separate butyraldehyde from water. The water extracted from the decanter (Stream 21) is also recovered as purified water and the butyraldehyde extracted from the decanter (Stream 20) is then fed to the feedstock aqueous solution as extracting medium (Stream 11).

The feedstock aqueous solution of acetic acid having an acetic acid concentration from about 10 to about 50% wt. % which is provided under step a) was prepared as follows in view of obtaining a fully bio-based acetic acid product as illustrated in FIG. 1B and as detailed below:

Feeding an ethanol/water mixture containing from about 10 to 95 wt. % ethanol into a heating vessel (stream 1) and bringing the temperature of the ethanol/water mixture from about 160 to about 250° C. (steps i)-ii))

A water ethanol mixture inlet was fed into a heating vessel at a rate of 13.43 ton/h (41.5% Ethanol, 58.5% Water) at 25° C. and 1 bar and heated at a temperature of about 180° C. Supplying the heated ethanol/water mixture to a heterogeneous catalyst to a heterogeneous catalyst at a temperature from about 160 to about 250° C. (step iii) and supplying the heated ethanol/water mixture in presence of the heterogeneous catalyst with air, oxygen-enriched air, or oxygen flow with an amount of oxygen that is 0.5-1.25 times the molar content in ethanol of the mixture and raising the pressure of the reaction vessel to reach a pressure of 20-60 bar, typically 30-50 bar (stream 4) (step iv)

The heated mixture was transferred to a reaction vessel at a temperature of about 180° C. (stream 1a) and the heated mixture was supplied with compressed air to raise the pressure of the reaction vessel to 40 bar for at a temperature of 180° C. over a RuO₂-based catalyst bed to induce the formation of acetic acid.

Recovering the reaction products (stream 5) containing acetic acid, water, unreacted ethanol, traces of acetaldehyde, unreacted oxygen, nitrogen and minor impurities (e.g. acetyls, acetates) into a first flash evaporation vessel at 1-10 bar, typically 1-5 bar, and 12-25° C., typically 18-20° C. (step v)

The reaction products are collected as a stream of a 44.43 ton/h (14.1% acetic acid, 23.4% water, 7.57% ethanol, 0.54% acetaldehyde, 6.77% oxygen, 47.65% nitrogen) at 180° C. and 40 bar and sent to a turbine for depressurization and energy recovery and to a cooler. The stream then reaches 1 bar and 20° C. and is sent to a flash evaporation vessel for volatiles removal.

Removing of the volatiles from the reaction products (e.g. oxygen, nitrogen, and acetaldehyde) from the flash evaporation vessel (stream 6) and transferring the remaining liquid phase (Stream 7, mainly containing acetic acid, water and unreacted ethanol) to a first distillation column at 50-120° C., typically 75-102° C. for the removal of ethanol and traces of acetaldehyde (step vi)

The vapour stream (Stream 6) leaving the flash vessel at a rate of 25.28 ton/h stream contained 0.33% acetic acid, 1.27% water, 2.05% ethanol, 0.73% acetaldehyde, 11.9% oxygen and 83.7% nitrogen at 20° C. and 1 bar.

The liquid collected from the outlet stream of the flash vessel (Stream 7) at a rate of 19.14 ton/h stream contained 32.2% acetic acid, 52.6% water, 14.9% ethanol and 0.30% acetaldehyde at 20° C. and 1 bar and was sent to a first distillation column for the removal of unreacted ethanol and acetaldehyde traces at a temperature of 75-102° C. under atmospheric temperature. The number of selected theoretical stages is 14, the feed stage is 7, and the distillate molar component recovery of ethanol is 99.99% (stream 8).

Recovering the aqueous solution from the distillation step (stream 10), wherein said aqueous solution comprises acetic acid having at a concentration from about 10 to about 50% wt. %, typically 30-40 wt. % (step vii)

The product at the bottom of the flash vessel (Stream 10) was collected at a rate of 15.68 ton/h of 39.32% acetic acid in water at 101.4° C. and 1 bar.

Subjecting the distillates (stream 8) obtained from the distillation step to an evaporation step to recover the unreacted ethanol in water with a concentration of 50-99%, typically 70-85% (stream 2) (step viii)

The distillate (Stream 8) is obtained at a rate of 3.460 ton/h stream (16.1% water and 82.3% ethanol and 1.64% of acetaldehyde) at 79.01° C. and 1 bar and is cooled down to 20° C. and sent to a second flash vessel, which recovers unreacted ethanol, water and traces of acetaldehyde (Stream 2) as a liquid at a rate of 3.461 ton/h (16.1% water, 82.2% ethanol, 1.64% acetaldehyde) which is then mixed with water ethanol mixture inlet (stream 1).

The two gaseous outlet streams (streams 6 and 9) from the Volatile Removal and Flash Separator, which are constituted mainly of oxygen and nitrogen, are sent to treatment before being released into the environment.

### Example 2: Comparison of Methods for the Preparation of Bio-Based Acetic Acid (Comparative Method, not from the Invention)

A comparison was carried out between a method for the preparation of bio-based acetic acid from the invention and another method using ethyl acetate.

The method of the invention was carried out as described above by providing a batch of 50 mL of diluted acetic acid aqueous solution (1.67% acetic acid, 0.28% acetaldehyde, 5.63% ethanol and 92.4% water), with >2 ppm of iron, and supplying 100 grams of Butyraldehyde to the said diluted acetic acid aqueous solution. In separation funnels, a diluted acetic acid aqueous solution is introduced, followed by the addition of 100 g of butyraldehyde. The mixtures are thoroughly shaken to replicate extraction (shake test) and left to separate for at least 15 minutes. Samples from both phases are then collected using 1 ml syringes after measuring the mass and volume of the two phases.

As comparison, the same method was applied except that 100 g of ethyl acetate are introduced instead of Butyraldehyde.

As can be seen, butyraldehyde used as an extraction agent, performs better extraction than ethyl acetate providing 5% wt. % more extraction efficiency than ethyl acetate as shown in Table 1 below:

TABLE 1

| | ethyl acetate as | butyraldehyde as |
|---|---|---|
| | extracting agent | extracting agent |
| Acetic acid concentration | 0.18M | 0.19M |
| Iron content in Water phase | >2 ppm | 0.5-2 ppm |

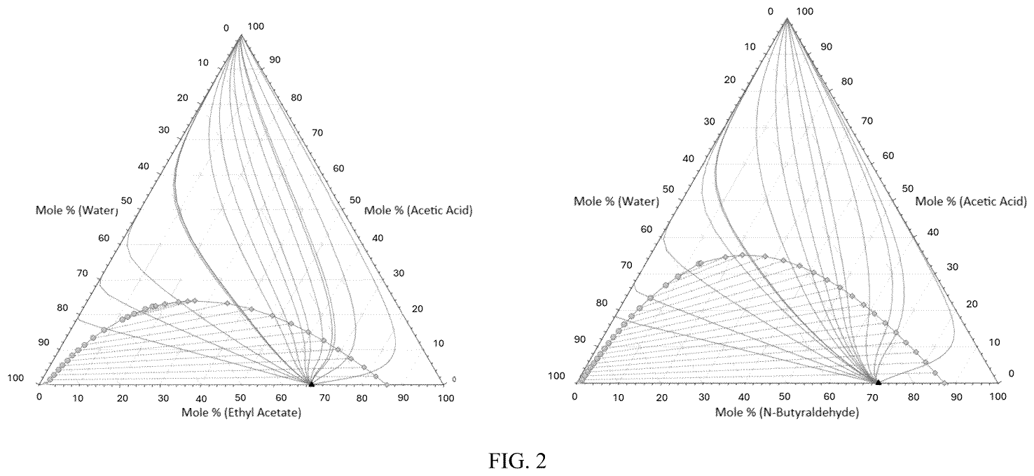

Further, the use of an extraction medium comprising butyraldehyde allows using higher concentrations of acetic acid in the feed-stock solution than with ethyl acetate since butyraldehyde shows lower solubility in water and has a broader miscibility gap (FIG. 2). Additionally, butyraldehyde avoids the extraction of transition metals, like Copper, Iron, and Nickel (not transferred to the extract stream (stream 12) which are not detected in the extraction phase by a stripe test as opposed as when using ethyl acetate for the extraction.

As can be seen in the enclosed ternary diagrams, butyraldehyde's azeotrope and solvent boiling points are lower than for ethyl Acetate in FIG. 2 which facilitates the distillation step to separate the solvent from acetic acid.

Further, butyraldehyde can be obtained via bioethanol, yielding a fully bio-based process.

### Example 3: Schematic Example of an Industrial Set-Up for a Method for the Preparation of Bio-Based Acetic Acid According to the Invention

An example of a full set-up for implementing a method of the invention was simulated as illustrated in FIGS. 3B+3A to support the scalability of the method of the invention. The simulation was set up in CHEMCAD™ to prove the scalability and competitiveness of the process of the invention compared to existing processes.
Step i): Fresh water and ethanol mixture containing from about 10 to 80 wt. % ethanol is fed into a heating system (stream 1).
Step ii): The stream is then heated to the reaction temperature from about 160° C. to about 250° C. (e.g. 180° C.).
Steps iii) and vi): The heated stream (stream 1a) is mixed with compressed air and transferred to a reaction vessel. The air was compressed via a multi-stage compression & cooling element up to 40 bar.
Step v): The reaction products are recovered. The reaction yields 60% conversion and 95% selectivity. The outlet stream (stream 5) is sent through a turbine and a heat exchanger to depressurize to 1 bar and cool down to 20° C.
Step vi): The cooled and depressurized stream 5 is then sent to a flash evaporation vessel for removing the volatiles where a first gas/liquid separation allows for a primary separation of unreacted oxygen, acetaldehyde and inert nitrogen and transferring the remaining liquid phase (stream 7) to a distillation column to remove traces of acetaldehyde and unreacted ethanol as a distillate (stream 8).
Step vii): The liquid outlet from the distillation column (stream 7) is an aqueous acetic acid solution comprising acetic acid at a concentration from about 10 to about 50% wt. % that is fed to the extraction method of the invention (step a), after being cooled down to 25° C. (stream 10).
Step viii): The distillate (stream 8) is sent to a second flash vessel where a gas/liquid separation occurs step to recover the unreacted ethanol in water. The liquid stream from the flash vessel constitutes the water/ethanol recycle stream (stream 2) which is then mixed with the feed of Fresh water and ethanol mixture (stream 1) whereas the gas is sent along with the outlet stream (stream 9) from the first flash separation vessel to off-gas treatment.
Step b): The feedstock aqueous solution comprising acetic acid at a concentration from about 10 to about 50% wt. % (stream 10) is supplied with an extracting medium containing butyraldehyde (stream 11) at the bottom of an extraction column to extract acetic acid to the organic phase (water-poor phase)) while the water phase is extracted from the extraction column for further purification (stream 13). The extracting medium containing butyraldehyde comprises a butyraldehyde recycle stream (stream 18), containing some water.
Steps c)-d): The organic phase, or solvent-rich phase (stream 12) is heated to 35° C. and sent to an azeotropic distillation column, where glacial acetic acid is recovered as the bottom product (stream 14). The distillate (stream 15), constituted mainly by butyraldehyde and traces of water is sent to a decanter. Here the increase in residence time allows for better separation of the two immiscible phases (solvent phase and water phase). The solvent-rich stream (stream 18) is sent back to the extraction column (stream 11), whereas the water-rich stream (stream 19) is sent, along with the water-rich stream from the extraction column (stream 13), to the water purification column.
Step e): The water rich phase recovered after azeotropic distillation is recovered as a pure water from the bottom of the water purification column (stream 16) while the solvent impurities are separated as a distillate product (stream 17). The distillate is sent to a second decanter where a stronger separation between the two immiscible phases is obtained. The solvent-rich phase (stream 20) is recycled back to the extraction column (stream 11) while the water-rich phase (stream 21) is mixed with the bottom product of the water purification column (stream 16). This stream constitutes the water that can be recycled back to the inlet of the process as fresh water feed.

1. A method for the preparation of bio-based acetic acid according to the ASTM D6866 standard comprising the steps of:
a) providing a feedstock aqueous solution of acetic acid having an acetic acid concentration from about 1 to about 50 wt. % into an extraction vessel;
b) supplying the feedstock aqueous solution with an extracting medium containing butyraldehyde to separate from the obtained mixture between the extracting medium and the aqueous solution of acetic acid, a water-poor phase rich in butyraldehyde and a water-rich phase;
c) subjecting the extracted water-poor phase rich in butyraldehyde and acetic acid to an azeotropic distillation to separate acetic acid from butyraldehyde;
d) recovering anhydrous acetic acid by distillation of the water-poor phase rich in butyraldehyde;
e) recovering purified water by distillation of the water-rich phase.

2. The method according to claim 1, wherein butyraldehyde is provided at a mass ratio with the feedstock mass from 1 to 2.

3. The method according to claim 1, wherein the obtained mixture between the extracting medium and the aqueous solution of acetic acid is subjected to an extraction in the extraction vessel at a temperature from about 10-35° C. to separate water-poor phase rich in butyraldehyde and acetic acid from a water-rich phase.

4. The method according to claim 1, wherein the extracted water-poor phase rich in butyraldehyde is subjected to an azeotropic distillation at a temperature from about 50-150° C. under atmospheric pressure to separate acetic acid from butyraldehyde.

5. The method according to claim 1, wherein the bottom liquid phase obtained as bottom product from the azeotropic distillation column contains anhydrous acetic acid with an amount from about 99.8 and 99% wt. of said phase.

6. The method according to claim 1, wherein the distillate containing butyraldehyde extracted from the azeotropic distillation contains from 1 to 20 wt. % water and 80 to 99 wt. % butyraldehyde.

7. The method according to claim 1, wherein the water-rich phase extracted from the extraction vessel is supplied to a water purification system to recover purified water.

8. The method according to claim 1, wherein feedstock aqueous solution of acetic acid having an acetic acid concentration from about 1 to about 90 wt. % is prepared by a method comprising the following steps:
feeding an ethanol/water mixture containing from about 10 to 95 wt. % ethanol into a reaction vessel;
subjecting the ethanol/water mixture to aerobic oxidation under a temperature from about 160 to about 250° C. at a pressure of 20-60 bar in presence of a heterogeneous catalyst;
recovering the reaction products containing acetic acid, water, unreacted ethanol, traces of acetaldehyde, unreacted oxygen, nitrogen and impurities;
removing the volatiles by evaporation and subjecting the remaining liquid phase to a distillation step for the removal of ethanol and traces of acetaldehyde;
recovering the aqueous solution from the distillation step, wherein said aqueous solution comprises acetic acid having at a concentration from about 1 to about 90 wt. %; and
subjecting the distillates obtained from the distillation step to an evaporation step to recover the unreacted ethanol in water.

9. The method according to claim 8, wherein the ethanol/water mixture is a bioethanol mixture obtainable by bioethanol production processes.

10. The method according to claim 8, wherein the aerobic oxidation comprises supplying the reaction vessel with air flow with an amount of oxygen that is 0.5-1 times the molar content in ethanol of the mixture and raising the pressure of the reaction vessel to reach a pressure of 20-60 bar.

11. The method according to claim 8, said method comprising the following steps:
i) feeding an ethanol/water mixture containing from about 10 to 95 wt. % ethanol into a heating vessel (stream 1);
ii) bringing the temperature of the ethanol/water mixture to a temperature from about 160 to about 250° C.;
iii) supplying the heated ethanol/water mixture to a heterogeneous catalyst at a temperature from about 160 to about 250° C.;
iv) supplying the heated ethanol/water mixture in presence of the heterogeneous catalyst with air, oxygen-enriched air, or oxygen flow with an amount of oxygen that is 0.5-1.25 times the molar content in ethanol of the mixture and raising the pressure of the reaction vessel to reach a pressure of 20-60 bar (stream 4) to trigger an aerobic oxidation of the heated ethanol/water mixture;
v) recovering the reaction products (stream 5) containing acetic acid, water, unreacted ethanol, traces of acetaldehyde, unreacted oxygen, nitrogen and minor impurities;
vi) removing the volatiles by evaporation (stream 6) and transferring the remaining liquid phase (Stream 7) containing acetic acid, water and unreacted ethanol to a distillation step for the removal of ethanol and traces of acetaldehyde;
vii) recovering the aqueous solution from the distillation step (stream 10), wherein said aqueous solution comprises acetic acid having at a concentration from about 1 to about 90 wt %; and
viii) subjecting the distillates (stream 8) obtained from the distillation step to an evaporation step to recover the unreacted ethanol in water.

12. The method according to claim 8, wherein the reaction products containing acetic acid, water, unreacted ethanol, traces of acetaldehyde, unreacted oxygen, nitrogen and impurities are supplied into a first evaporation vessel at 1-10 bar at a temperature of 12-25° C. from which the volatiles are removed by evaporation at a temperature from about 50-120° C.

13. The method according to claim 8, wherein the distillates from the distillation step are transferred to a second flash evaporation at 1-10 bar and 12-25° C. to recover the unreacted ethanol in water with a concentration of 50-99 wt. % which can be recycled into the reaction vessel.

14. The method according to claim 1, wherein said method is carried out in a continuous or semi-continuous flow.

15. The method according to claim 8, wherein the heterogeneous catalyst is ruthenium oxide-based, gold-based, platin-based, palladium-based, tungsten oxide-based or vanadium oxide-based.

16. A bio-acetic acid with after synthesis levels in formaldehyde and formic acid below 10 ppm before any purification with a C¹⁴ pMC equal to the contemporary ASTM reference for bio-based materials.
